(19) United States
(12) Patent Application Publication
(10) Pub. No.: US 2024/0238246 A1
(43) Pub. Date: Jul. 18, 2024

(54) METHODS FOR PREVENTING OR SLOWING THE PROGRESSION OF COGNITIVE DECLINE OR IMPAIRMENT IN SUBJECTS

(71) Applicants: AGENEBIO, INC., Baltimore, MD (US); The Johns Hopkins University, Baltimore, MD (US)

(72) Inventors: Richard Mohs, Baltimore, MD (US); Michela Gallagher, Baltimore, MD (US); Arnold Bakker, Baltimore, MD (US)

(21) Appl. No.: 18/411,753

(22) Filed: Jan. 12, 2024

Related U.S. Application Data
(60) Provisional application No. 63/545,348, filed on Oct. 23, 2023.
(60) Provisional application No. 63/439,035, filed on Jan. 13, 2023.

(51) Int. Cl. A61K 31/4015; A61K 9/20; A61P 25/28

(52) U.S. Cl. CPC A61K 31/4015; A61K 9/2009; A61K 9/2013; A61K 9/2054; A61P 25/28

(57) ABSTRACT

Methods for preventing or slowing the progression of cognitive impairment or preventing the development or reducing the rate of cognitive decline. Methods for slowing the volumetric atrophy or reduction of a subregion of the medial temporal lobe. Methods for slowing the volumetric atrophy or reduction of the entorhinal cortex (ERC). Methods for slowing the volumetric atrophy or reduction of the transentorhinal cortex (BA35). The methods comprise administering to an APOE4 non-carrier subject one or more of levetiracetam, brivaracetam or seletracetam, or a pharmaceutically acceptable salt thereof, or administering a pharmaceutical composition comprising levetiracetam, brivaracetam or seletracetam or a pharmaceutically acceptable salt thereof and a pharmaceutically acceptable carrier.

### RELATED APPLICATIONS

This application claims the benefit of and priority from U.S. Provisional Application No. 63/439,035, filed Jan. 13, 2023, and U.S. Provisional Application No. 63/545,348, filed Oct. 23, 2023, each of which applications is incorporated herein by reference in its entirety.

### STATEMENT OF GOVERNMENT SUPPORT

The subject matter of this disclosure was made with government support under Grant No. R01AG061091 awarded by the National Institutes of Health (NIH), and in particular, its National Institute on Aging (NIA) division, an agency of the United States Government. The United States Government has certain rights in the subject matter of this disclosure.

### FIELD OF THE DISCLOSURE

This disclosure relates to methods for preventing or slowing the progression of cognitive impairment or preventing the development or reducing the rate of cognitive decline in a subject who is an APOE4 non-carrier, e.g., is not homozygous or heterozygous for APOE4. In some aspects, this disclosure relates to methods for delaying the reduction in volume, the shrinking of the thickness, or the atrophy of a subregion of the medial temporal lobe (e.g., entorhinal cortex (ERC) or transentorhinal cortex (BA35)), wherein the reduction in volume, shrinkage, or atrophy is characteristic of, and a biomarker for, the progression of cognitive impairment. In some aspects, this disclosure relates to methods for reducing the rate of the reduction in volume, the shrinking of the thickness, or the atrophy of a subregion of the medial temporal lobe (e.g., entorhinal cortex (ERC) or transentorhinal cortex (BA35)), wherein the rate of the reduction in volume, shrinkage, or atrophy is characteristic of, and a biomarker for, the progression of cognitive impairment.

In some aspects of this disclosure, the APOE4 non-carrier may display or present with cognitive performance within the normal range for the subject's age. In other aspects, the APOE4 non-carrier may present with preclinical cognitive impairment, i.e., displaying or presenting with cognitive performance somewhat below the normal range for the subject's age, with pre-mild cognitive impairment (pre-MCI), with mild cognitive impairment, with mild cognitive impairment (MCI) due to Alzheimer's disease (AD), with prodromal AD or with amnestic MCI (aMCI). In some aspects of this disclosure, the APOE4 non-carrier may have a risk of developing or presenting with cognitive impairment associated with various other CNS disorders. In some aspects of this disclosure, the APOE4 non-carrier presents with a reduced volume, shrinkage or atrophy of a subregion of the medial temporal lobe (e.g., entorhinal cortex (ERC) or transentorhinal cortex (BA35)), the non-carrier being selected from the group of subjects displaying or presenting with a volume in a subregion of the medial temporal lobe that is less than normal and subjects displaying or presenting with cognitive performance that is within or in some aspects somewhat below the normal range for the subject's age. The methods of this disclosure comprise administering to the subject one or more of levetiracetam, brivaracetam or seletracetam, or a pharmaceutically acceptable salt thereof, or a pharmaceutical composition comprising levetiracetam, brivaracetam or seletracetam or pharmaceutical salt thereof and a pharmaceutically acceptable carrier.

In some aspects of this disclosure the APOE4 non-carrier has one or more risks that are predictive of or associated with developing cognitive decline or cognitive impairment or the progression of the decline or impairment. Some of such risks are associated with aging. Others are genetic risks associated with genomic variants, mutations, or polymorphs, or associated with a change in the expression of genes associated with cognitive decline or impairment. In some aspects of this disclosure, the genetic risk factor is not APOE4. In some aspect the risk is a reduction in volume, shrinkage or atrophy of a subregion of the medial temporal lobe (e.g., entorhinal cortex (ERC) or transentorhinal cortex (BA35)).

### BACKGROUND OF THE DISCLOSURE

Cognitive ability may decline as a normal consequence of aging or may be associated with changes in hippocampal activity, e.g. hyperactivity, with genomic variations, mutations, or polymorphs, with the development of cognitive impairment or decline in the context of CNS diseases and disorders, such as Alzheimer's disease (AD) or with changes in the volume of, or the shrinkage or atrophy of a subregion of the medial temporal lobe (e.g., entorhinal cortex (ERC) or transentorhinal cortex (BA35)). Although subjects may present or display with cognitive performance in the normal range for their age, they may still be at risk of developing cognitive impairment or decline.

Post-mortem studies of patients with AD dementia have provided insight into the spatial and temporal progression of AD pathology in the brain, revealing that the formation of neurofibrillary tangles (NFTs) in the initial stages of AD pathology occurs in the entorhinal cortex (ERC), specifically in the transentorhinal cortex (TEC or BA35), which serves as the transition between the lateral portions of the ERC and the perirhinal cortex (Braak et al., Acta Neuropathologica, 112(4), 389-404 (2006); Braak & Braak, Acta Neuropathologica, 80(5), 479-486 (1990); Kaufman et al., Acta Neuropathologica, 136(1), 57-67 (2018)). Tau accumulation in the TEC is also common by age 60, even among cognitively normal older adults (Maass et al., Journal of Neuroscience, 38(3), 530-543 (2018)). Tau pathology then spreads through other of the medial temporal lobe regions (MTL; Braak et al., 2006), which have a role in episodic memory functioning (Dickerson & Eichenbaum, Neuropsychopharmacology, 35(1), Article 1 (2010)). The localization and spreading of tau co-occurs temporally and spatially with the progression of neural degeneration.

The entorhinal cortex has shown disease related decline in cortical volume in cross sectional and longitudinal studies. For example, Fan et al. reported atrophy in the left entorhinal cortex in patients who are cognitively normal but have subjective memory decline (Fan et al., Hum Brain Mapp., 39(6):2549-2562 (2018)). Tran et al. reported significantly reduced volume localized to a bilateral subregion of the entorhinal cortex consistent with the TEC in patients with mild cognitive impairment (Tran et al., Neurobiol Aging, 112:151-160 (2022)). Tward et al. reported significant atrophy localized to the transentorhinal cortex in patients with mild cognitive impairment and Kulason et al. showed that changes in volume and thickness of the entorhinal and TEC can be observed in participants 8-11 and 9-14 years before a diagnosis of mild cognitive impairment is made (Tward et al., Alzheimers Dement (Amst), 14(9):41-50 (2017); Kulason et al. Front Neurosci., 14:804 (2020)).

Although APOE4 is one of the more prominent genetic risk factors for developing cognitive decline, including AD, it is neither necessary nor sufficient. Accordingly, a person with one or two copies of the APOE4 polymorph, i.e. an APOE4 carrier, may never experience cognitive decline or suffer from AD or other dementias, and conversely, a person who is an APOE4 non-carrier may suffer cognitive decline and the associated diseases and disorders. The focus, however, in many treatment studies have been on subjects that are APOE4 carriers, i.e., having one of both APOE4 alleles.

Indeed, no FDA-approved therapy exists for APOE4 non-carriers who display or present with cognitive performance within the normal range for the subject's age but who may be at risk due to aging, shrinkage or atrophy of a subregion of the medial temporal lobe (e.g., entorhinal cortex (ERC) or transentorhinal cortex (BA35)), or various genetic risk factors or CNS disorders for developing or progressing to cognitive impairment or cognitive decline. Nor, outside of amyloid targeting biologics, do any treatments exist that are said to specifically prevent or slow the progression of cognitive impairment or prevent the development or reduce the rate of cognitive decline in subjects suffering from aMCI or MCI due to AD. Such biologics are also expensive, are administered intravenously, and require careful monitoring for potential side effects, e.g., brain bleeding.

There is, therefore, a need for effective clinical and therapeutic measures for (1) preventing or slowing the progression of cognitive impairment; (2) preventing the development or reducing the rate of cognitive decline; or (3) delaying or reducing the rate of volumetric atrophy or shrinkage of a subregion of the medial temporal lobe (e.g., entorhinal cortex (ERC)) in APOE4 non-carriers who display or present with cognitive performance within the normal range for the subject's age but who may be at risk for developing or progressing to cognitive impairment or cognitive decline as well as in APOE4 non-carriers who display the early stages of cognitive impairment and decline, for example, subjects who present with symptoms associated with pre-MCI, aMCI or MCI due to AD whether or not the pre-MCI, aMCI or MCI is associated with aging. There is also a need for less expensive and safer therapeutic options for reducing and delaying the progression of such cognitive impairment.

### BRIEF SUMMARY OF THE DISCLOSURE

This disclosure relates to methods for preventing or slowing the progression of cognitive impairment or preventing the development or reducing the rate of cognitive decline in APOE4 non-carriers displaying or presenting with cognitive performance within the normal range for the subject's age. In other aspects, this disclosure relates to methods for preventing or slowing the progression of cognitive impairment or preventing the development or reducing the rate of cognitive decline in APOE4 non-carriers displaying or presenting with cognitive performance below the normal range for the subject's age.

In other aspects, the APOE4 non-carrier may present with preclinical cognitive impairment, i.e., displaying or presenting with cognitive performance somewhat below the normal range for the subject's age, with pre-mild cognitive impairment (pre-MCI), with mild cognitive impairment, with mild cognitive impairment (MCI) due to Alzheimer's disease (AD), with prodromal AD or with amnestic MCI (aMCI). In other aspects, this disclosure relates to methods for preventing or slowing the progression of cognitive impairment or preventing the development or reducing the rate of cognitive decline in an APOE4 non-carrier having a risk of developing or presenting with cognitive impairment associated with various other CNS disorders. In other aspects, this disclosure relates to methods for delaying or reducing the rate of volumetric atrophy or shrinkage of a subregion of the medial temporal lobe in an APOE4 non-carrier subject selected from the group of subjects displaying or presenting with a volume in a subregion of the medial temporal lobe that is less than normal and subjects displaying or presenting with cognitive performance that is within or in some aspects somewhat below the normal range for the subject's age. In other aspects, this disclosure relates to methods for delaying or reducing the rate of volumetric atrophy or shrinkage of the entorhinal cortex in an APOE4 non-carrier subject. The methods comprise administering to the subject one or more of levetiracetam, brivaracetam or seletracetam, or a pharmaceutically acceptable salt thereof, or a pharmaceutical composition comprising levetiracetam, brivaracetam or seletracetam or pharmaceutical salt thereof and a pharmaceutically acceptable carrier. In some embodiments of this disclosure, the methods comprise administering to the subject levetiracetam, or a pharmaceutically acceptable salt thereof, and a pharmaceutically acceptable carrier.

In some aspects of this disclosure the APOE4 non-carriers present with one or more risk factors predictive of or associated with the development of cognitive decline or cognitive impairment or the progression of the decline or impairment. Some of such risks are associated with aging. Others are genetic risks associated with genomic variants, mutations, or polymorphs, or associated with a change in the expression of genes associated with cognitive decline or impairment. In some aspects of this disclosure, the genetic risk factor is not APOE4. In some aspects, the risk is a reduction in volume, shrinkage or atrophy of a subregion of the medial temporal lobe (e.g., entorhinal cortex (ERC) or transentorhinal cortex (BA35)).

In some embodiments, the methods of this disclosure comprise administering to the subject one of more of levetiracetam, brivaracetam or seletracetam, or a pharmaceutically acceptable salt thereof, at a daily dose of 0.7-350 mg. In other aspects, the methods of this disclosure comprise administering to the subject a pharmaceutical composition comprising a daily dose of 0.7-350 mg of one or more of the levetiracetam, brivaracetam or seletracetam, or pharmaceutically acceptable salt thereof, and a pharmaceutically acceptable carrier. In some embodiments, the methods of this disclosure comprise administering to the subject levetiracetam or a pharmaceutically acceptable salt thereof, at a daily dose of 0.7-350 mg.

In some embodiments, the daily dose of the levetiracetam or seletracetam, or a pharmaceutically acceptable salt thereof, themselves or as part of a pharmaceutical composition, is 7-350 mg. In some embodiments, the daily dose of the brivaracetam, or pharmaceutically acceptable salt thereof, themselves or as part of a pharmaceutical composition, is 0.7-180 mg. In other embodiments, the daily dose of the levetiracetam or seletracetam, or pharmaceutically acceptable salt thereof, themselves or as part of a pharmaceutical composition, is 125-250 mg. In some embodiments, the daily dose of the levetiracetam or seletracetam, or pharmaceutically acceptable salt thereof, themselves or as part of a pharmaceutical composition, is 220 mg. In some embodiments, the daily dose of the levetiracetam or seletracetam, or pharmaceutically acceptable salt thereof, themselves or as part of a pharmaceutical composition, is 190 mg.

In some embodiments, the levetiracetam, brivaracetam or seletracetam or a pharmaceutically acceptable salt thereof or the pharmaceutically acceptable composition comprising one of more of them is formulated in one or more of an oral form, an extended-release form, a single-unit-dosage form or a once-a-day administration form. In some embodiments, the extended-release form is a controlled release form, a prolonged release form, a sustained release form, a delayed release form, or a slow-release form. In some aspects of this disclosure the extended-release form, the single-unit-dosage form and the once-a-day form are for oral administration. In some embodiments, the levetiracetam, brivaracetam or seletracetam or a pharmaceutically acceptable salt thereof, or the pharmaceutically acceptable composition comprising one of more of them is administered once daily. In some embodiments, the levetiracetam, brivaracetam or seletracetam or a pharmaceutically acceptable salt thereof, or the pharmaceutically acceptable composition comprising one of more of them is administered twice daily.

In some embodiments of this disclosure, the method comprises administering to the APOE4 non-carrier an oral, daily, single use-dosage-form of an extended release pharmaceutical composition comprising 220 mg of levetiracetam or pharmaceutically acceptable salt thereof, 280 mg-350 mg of hydroxypropyl methylcellulose, 1.2 mg-1.4 mg of colloidal silicon dioxide, 92.8 mg-119.2 mg of silicified microcrystalline cellulose, and 6.0 mg-6.7 mg of magnesium stearate. In other embodiments, the daily dose of the levetiracetam, or pharmaceutically acceptable salt thereof, in the extended-release pharmaceutical composition is 220 mg and the pharmaceutical composition further comprises 280 mg of hydroxypropyl methylcellulose, 1.2 mg of colloidal silicon dioxide, 92.8 mg of silicified microcrystalline cellulose, and 6.0 mg of magnesium stearate. In other embodiments, the daily dose of the levetiracetam, or pharmaceutically acceptable salt thereof, in the extended-release pharmaceutical composition is 220 mg and the pharmaceutical composition further comprises 347.5 mg of hydroxypropyl methylcellulose, 1.4 mg of colloidal silicon dioxide, 119.2 mg of silicified microcrystalline cellulose, and 6.7 mg of magnesium stearate. In other embodiments of the extended-release composition useful in the methods of this disclosure, the hydroxypropyl methylcellulose is hypromellose 2208. In other embodiments, the silicified microcrystalline cellulose is silicified microcrystalline cellulose SMCC 90.

In some embodiments of this disclosure, the method comprises administering to the APOE4 non-carrier an oral, daily, in some embodiments once per day, single use-dosage-form of an extended release pharmaceutical composition comprising 220 mg of levetiracetam, 280 mg-350 mg of hydroxypropyl methylcellulose, 1.2 mg-1.4 mg of colloidal silicon dioxide, 92.8 mg-119.2 mg of silicified microcrystalline cellulose, and 6.0 mg-6.7 mg of magnesium stearate. In other embodiments, the daily, or once per day, dose of the levetiracetam in the extended-release pharmaceutical composition is 220 mg and the pharmaceutical composition further comprises 280 mg of hydroxypropyl methylcellulose, 1.2 mg of colloidal silicon dioxide, 92.8 mg of silicified microcrystalline cellulose, and 6.0 mg of magnesium stearate. In other embodiments, the daily, or once per day, dose of the levetiracetam in the extended-release pharmaceutical composition is 220 mg and the pharmaceutical composition further comprises 347.5 mg of hydroxypropyl methylcellulose, 1.4 mg of colloidal silicon dioxide, 119.2 mg of silicified microcrystalline cellulose, and 6.7 mg of magnesium stearate. In other embodiments of the extended-release composition useful in the methods of this disclosure, the hydroxypropyl methylcellulose is hypromellose 2208. In other embodiments, the silicified microcrystalline cellulose is silicified microcrystalline cellulose SMCC 90.

In some embodiments of this disclosure, the method comprises administering to the subject a daily dose of a pharmaceutical composition comprising 190 mg of levetiracetam 300 mg of hydroxypropyl methylcellulose, 1.2 mg of colloidal silicon dioxide, 102.8 mg of silicified microcrystalline cellulose or anhydrous dicalcium phosphate, and 6 mg of magnesium stearate. In other embodiments, the hydroxypropyl methylcellulose is hypromellose 2208. In other embodiments, the silicified microcrystalline cellulose is silicified microcrystalline cellulose SMCC 90.

In some embodiments of this disclosure, the method comprises administering to the subject a daily, or once per day, dose of a pharmaceutical composition comprising 190 mg of levetiracetam, 300 mg of hydroxypropyl methylcellulose, 1.2 mg of colloidal silicon dioxide, 102.8 mg of silicified microcrystalline cellulose or anhydrous dicalcium phosphate, and 6 mg of magnesium stearate. In other embodiments, the hydroxypropyl methylcellulose is hypromellose 2208. In other embodiments, the silicified microcrystalline cellulose is silicified microcrystalline cellulose SMCC 90.

In some embodiments of this disclosure, the method comprises administering a pharmaceutical composition comprising an oral, daily, single use-dosage form of an extended release composition comprising levetiracetam, or pharmaceutically acceptable salt thereof, wherein the administration provides a steady state plasma concentration of levetiracetam in the APOE4 non-carrier of between 1.9 μg/mL and 4.4 μg/mL within 3 hours after administration and extending for at least 8 hours of a 24-hour period after said administration. In some embodiments of this disclosure, the method comprises administering a pharmaceutical composition comprising an oral, once per day, single use-dosage form of an extended release composition comprising levetiracetam, or pharmaceutically acceptable salt thereof, wherein the administration provides a steady state plasma concentration of levetiracetam in the APOE4 non-carrier of between 1.9 μg/mL and 4.4 μg/mL within 3 hours after administration and extending for at least 8 hours of a 24-hour period after said administration. In some embodiments of this disclosure, the method comprises administering a pharmaceutical composition comprising an oral, once per day, single use-dosage form of an extended release composition comprising levetiracetam wherein the administration provides a steady state plasma concentration of levetiracetam in the APOE4 non-carrier of between 1.9 μg/mL and 4.4 μg/mL within 3 hours after administration and extending for at least 8 hours of a 24-hour period after said administration. In some embodiments, the extended-release pharmaceutical composition provides a steady state plasma concentration of levetiracetam in the APOE4 non-carrier of between 1.9 μg/mL and 4.4 μg/mL within 2 hours after administration and extending for at least 13 hours of a 24-hour period after administration. In some embodiments, the extended-release pharmaceutical composition provides a steady state plasma concentration of levetiracetam in the APOE4 non-carrier of between 1.9 μg/mL and 4.4 μg/mL within 1 hour after said administration and extending for at least 13 hours of a 24-hour period after said administration. In other embodiments, the extended-release pharmaceutical composition provides a steady state plasma concentration of levetiracetam in the APOE4 non-carrier of between 1.9 μg/mL and 4.4 μg/mL within 1 hour after administration and extending for at least 13 to 16 hours of a 24-hour period after administration (See FIG. 2 and WO2016191288, which is incorporated by reference herein in its entirety).

In some embodiments of this disclosure, the APOE4 non-carrier to be treated is at risk of developing cognitive decline or cognitive impairment, wherein the risk is associated with aging. In some embodiments, the risk is associated with a reduction or shrinkage in the volume of subregion of the medial temporal lobe (e.g., entorhinal cortex (ERC) or transentorhinal cortex (BA35)). In other aspects, this disclosure relates to methods for preventing or slowing the progression of cognitive impairment or preventing the development or reducing the rate of cognitive decline in APOE4 non-carriers displaying or presenting with cognitive performance below the normal range for the subject's age. In some embodiments of this disclosure, the APOE4 non-carrier may present with preclinical mild cognitive impairment, i.e., displaying or presenting with cognitive performance somewhat below the normal range for the subject's age. In other aspects, this disclosure relates methods for delaying or reducing the rate of volumetric atrophy or shrinkage of a subregion of the medial temporal lobe (e.g., entorhinal cortex (ERC) or transentorhinal cortex (BA35)) in an APOE4 non-carrier subject, selected from the group of subjects displaying or presenting with a volume in a subregion of the medial temporal lobe that is less than normal and subjects displaying or presenting with cognitive performance that is within or in some aspects somewhat below the normal range for the subject's age. In other aspects, this disclosure relates to methods for delaying or reducing the rate of volumetric atrophy of the entorhinal cortex (ERC) in an APOE4 non-carrier subject. In some embodiments of this disclosure, the APOE4 non-carrier to be treated is at risk for developing volumetric atrophy of a subregion of the medial temporal lobe (e.g., entorhinal cortex (ERC) or transentorhinal cortex (BA35)). In some embodiments of this disclosure, the APOE4 non-carrier to be treated is at risk for developing volumetric atrophy of the entorhinal cortex (ERC). In some embodiments of this disclosure, the APOE4 non-carrier to be treated is at risk of developing cognitive decline or cognitive impairment, wherein the risk is associated with genomic variants, mutations, or polymorphs, or associated with a change in the expression of genes associated with cognitive decline or impairment. In some embodiments of this disclosure, the APOE4 non-carrier to be treated is at risk of developing cognitive decline or cognitive impairment, wherein the risk is associated with pre-MCI. In some embodiments of this disclosure, the APOE4 non-carrier to be treated is at risk of developing cognitive decline or cognitive impairment, wherein the risk is associated with aMCI. In some embodiments of this disclosure, the APOE4 non-carrier to be treated is at risk of developing cognitive decline or cognitive impairment, wherein the risk is associated with MCI. In some embodiments of this disclosure, the APOE4 non-carrier to be treated is at risk of developing cognitive decline or cognitive impairment, wherein the risk is associated with MCI due to Alzheimer's disease (AD) or prodromal AD.

One aspect of the disclosure relates to use of one or more of levetiracetam, brivaracetam or seletracetam or a pharmaceutical composition of any of them in the manufacture of a medicament for preventing or slowing the progression of cognitive impairment or preventing the development or reducing the rate of cognitive decline in a APOE4 non-carrier who with preclinical cognitive impairment, i.e., displaying or presenting with cognitive performance somewhat below the normal range for the subject's age. In other aspects, this disclosure relates to use of one or more of levetiracetam, brivaracetam or seletracetam or a pharmaceutical composition of any of them in the manufacture of a medicament for delaying or reducing the rate of volumetric atrophy of a subregion of the medial temporal lobe (e.g., entorhinal cortex (ERC) or transentorhinal cortex (BA35)) in an APOE4 non-carrier subject, selected from the group of subjects displaying or presenting with a volume in a subregion of the medial temporal lobe that is less than normal and subjects displaying or presenting with cognitive performance that is within or in some aspects somewhat below the normal range for the subject's age. In other aspects, this disclosure relates to use of one or more of levetiracetam, brivaracetam or seletracetam or a pharmaceutical composition of any of them in the manufacture of a medicament for delaying or reducing the rate of volumetric atrophy or shrinkage of the entorhinal cortex (ERC) in an APOE4 non-carrier subject.

In some embodiments, as described above, the APOE4 non-carrier is at risk for developing cognitive impairment or decline or the progression of the impairment or decline. In some embodiments, as described above, the APOE4 non-carrier is at risk for developing cognitive decline as a consequence of progression of volumetric atrophy of a subregion of the medial temporal lobe (e.g., entorhinal cortex (ERC) or transentorhinal cortex (BA35)). In some embodiments, as described above, the APOE4 non-carrier is at risk for developing volumetric atrophy of the entorhinal cortex (ERC). In some embodiments of this disclosure, the APOE4 non-carrier to be treated is at risk of developing cognitive decline or cognitive impairment, wherein the risk is associated with genomic variants, mutations, or polymorphs, or associated with a change in the expression of genes associated with cognitive decline or impairment. In some embodiments of this disclosure, as described above, the risk is associated with one or more of pre-MCI, MCI, aMCI, MCI due to AD, prodromal AD or other CNS disorder.

In other embodiments, this disclosure relates to the use of levetiracetam, brivaracetam or seletracetam or a pharmaceutical composition of any of them for preventing or slowing the progression of cognitive impairment or preventing the development or reducing the rate of cognitive decline in the APOE4 non-carrier displaying or presenting with cognitive performance within the normal range for the subject's age. In other aspects, this disclosure relates to the use of levetiracetam, brivaracetam or seletracetam or a pharmaceutical composition of any of them for delaying or reducing the rate of volumetric atrophy or shrinkage of a subregion of the medial temporal lobe (e.g., entorhinal cortex (ERC) or transentorhinal cortex (BA35)) in an APOE4 non-carrier subject, selected from the group of subjects displaying or presenting with a volume in a subregion of the medial temporal lobe that is less than normal and subjects displaying or presenting with cognitive performance that is within or in some aspects somewhat below the normal range for the subject's age. In other aspects, this disclosure relates to the use of one or more of levetiracetam, brivaracetam or seletracetam or a pharmaceutical composition of any of them for delaying or reducing the rate of volumetric atrophy of the entorhinal cortex (ERC) in an APOE4 non-carrier subject. In some embodiments, as described above, the APOE4 non-carrier is at risk for developing cognitive impairment or decline or the progression of the impairment or decline. In some embodiments, the APOE4 non-carrier is at risk for developing cognitive decline or cognitive impairment, wherein the risk is associated with the progression of volumetric atrophy of a subregion of the medial temporal lobe (e.g., entorhinal cortex (ERC) or transentorhinal cortex (BA35)). In some embodiments, as described above, the APOE4 non-carrier is at risk for developing volumetric atrophy of the entorhinal cortex (ERC). In some embodiments of this disclosure, the APOE4 non-carrier to be treated is at risk of developing cognitive decline or cognitive impairment, wherein the risk is associated with genomic variants, mutations, or polymorphs, or associated with a change in the expression of genes associated with cognitive decline or impairment. In some embodiments, as described above, the APOE4 non-carrier is at risk for developing cognitive impairment or decline or the progression of the impairment or decline. In some embodiments of this disclosure, as described above, the risk is associated with one or more of pre-MCI, MCI, aMCI, MCI due to AD, prodromal AD or other CNS disorder.

### BRIEF DESCRIPTION OF THE DRAWINGS

FIG. 1 depicts the plasma concentrations of levetiracetam effective in treating cognitive impairment based on aged-impaired rat studies and a phase II study in aMCI patients. In one aspect of this disclosure, the effective plasma concentration is between 1.9 and 4.4 μg/mL. In another aspect, the effective plasma concentration is between 2.9 and 4.4 μg/mL. In another aspect, the effective plasma concentration is between 1.9 and 3.9 μg/mL.

FIG. 2 shows the steady state modeling of the PK profile of the 190 mg Tablet A of Table 1, indicating that this tablet affords a plasma concentration of levetiracetam of between 1.9 and 4.4 μg/mL.

FIG. 3 shows the steady state modeling of the PK profile of the 220 mg Tablet D of Table 2, indicating that this tablet affords a plasma concentration of levetiracetam of between 2.9 and 4.4 μg/mL.

FIG. 4 is a flow diagram of one embodiment of a process for manufacturing extended-release compositions of levetiracetam (e.g., the 190 mg and 220 mg tablets listed in Tables 1 and 2).

FIG. 5 provides the study design a multicenter, randomized, double-blind, placebo-controlled, 78-week, fixed-dose study evaluating a low-dose levetiracetam, 220 mg, extended-release tablet versus placebo as a treatment for slowing the progression of mild cognitive impairment (MCI) due to Alzheimer's disease (AD).

FIG. 6 provides a graphical representation of the Clinical Dementia Rating-Sum of Boxes (CDR-SB) results over the 78 weeks. CDR-SB scores were taken at baseline, 26 weeks, 52 weeks, and 78 weeks after levetiracetam (LEV) or placebo treatment including the difference of the two groups over time.

FIG. 7 provides a graphical representation of Functional Activities Questionnaire (FAQ) score results over the 78 weeks. CDR-SB scores were taken at baseline, 26 weeks, 52 weeks, and 78 weeks after levetiracetam (LEV) or placebo treatment including the difference of the two groups over time.

FIG. 8 provides a graphical representation of entorhinal cortex (ERC) volume at baseline and the ERC volume change after 78 weeks of levetiracetam (LEV) or placebo administration to APOE4 non-carriers.

FIG. 9 provides a graphical representation of Brodmann area 35 (BA35) volume at baseline and BA35 volume change after 78 weeks levetiracetam (LEV) or placebo administration to APOE4 non-carriers.

### DETAILED DESCRIPTION OF THE DISCLOSURE

Unless otherwise defined herein, scientific and technical terms used in this application shall have the meanings that are commonly understood by those of ordinary skill in the art. Generally, nomenclature used in connection with, and techniques of, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art. See, e.g., “Principles of Neural Science,” McGraw-Hill Medical, New York, N.Y. (2000); Motulsky, “Intuitive Biostatistics,” Oxford University Press, Inc. (1995); Lodish et al., “Molecular Cell Biology, 4th ed.,” W.H. Freeman & Co., New York (2000); Griffiths et al., “Introduction to Genetic Analysis, 7th ed.,” W.H. Freeman & Co., N.Y. (1999); Gilbert et al., “Developmental Biology, 6th ed.,” Sinauer Associates, Inc., Sunderland, MA (2000).

Chemistry terms used herein are used according to conventional usage in the art, as exemplified by “The McGraw-Hill Dictionary of Chemical Terms,” Parker S., Ed., McGraw-Hill, San Francisco, CA (1985).

All publications, patents and published patent applications referred to in this application are specifically incorporated by reference herein in their entirety. In case of conflict, the present specification, including its specific definitions, will control.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

“Including” is used to mean “including but not limited to”. “Including” and “including but not limited to” are used interchangeably.

“Patient,” “subject”, or “individual” are used interchangeably and refers to either a human or a non-human animal. Patient, subject, or individual may include mammals, such as humans, primates, livestock animals (including bovines, porcines, etc.), companion animals (e.g., canines, felines, etc.) and rodents (e.g., mice and rats). In some embodiments, the patient, subject, or individual is a human.

“Preventing” the development or progression of cognitive decline or impairment refers to affecting cognitive performance such that it does not decline or does not fall below that observed in the subject upon first presentation or diagnosis or delays such decline or performance.

“Slowing” the development or progression of cognitive decline refers to delaying the progression of cognitive decline in a subject. This may be determined by a physician using well known assessments of cognition or function (e.g., AD Composite Score (ADCOMS), the Alzheimer's Disease Assessment Scale-Cognitive Subscale (ADAS-Cog), Alzheimer's Disease Cooperative Study-Activities of Daily Living-MCI (ADCS-ADL-MCI), or one of more of the assessments referred to infra) or by comparison with untreated populations.

“Cognitive impairment” or “cognitive decline” refers to cognitive performance in subjects that is not as robust as the normal range expected in a subject of similar age. In some cases, cognitive performance is reduced by about 5%, about 10%, about 30%, or more, compared to normal range of cognitive performance expected in a subject of similar age. In some cases, “cognitive impairment” in subjects may refer to cognitive performance in subjects that is not as robust as the normal range that is expected in an aged-matched subject, or the performance of a young adult subject (e.g., subjects with mean scores for a given age in a test of cognitive performance).

“Pre-MCI” or “Pre-clinical MCI” refers to subjects who exhibit features of MCI on clinical examination but lack impairment on neuropsychological examination. Subjects may present with biomarkers other than APOE4 that identify risk for progression.

“APOE4 carrier” or “APOE4 positive subject” refers to a subject that carries one or both copies of the Apolipoprotein E 4 (APOE4) allele. APOE is a protein involved in the metabolism of fats in the body of mammals and is polymorphic, with three major alleles: APOE2, APOE3, and APOE4 with 25% of the general population carrying one copy of the APOE4 allele and 2-3% carrying both alleles. APOE4 carriers may be determined using genotyping techniques, including but not limited to, restriction fragment length polymorphism identification (RFLPI) of genomic DNA (See., e.g. Dai, S., Long, Y. (2015). In: Batley, J. (eds) Plant Genotyping. Methods in Molecular Biology, vol 1245. Humana Press, New York, NY., random amplified polymorphic detection (RAPD) of genomic DNA (See, e.g, Williams J G, Kubelik A R, Livak K J, Rafalski J A, Tingey S V. Nucleic Acids Res. 1990 Nov. 25; 18(22):6531-5.), amplified fragment length polymorphism detection (AFLPD) (See, e.g., Paun O, Schonswetter P. Methods Mol Biol. 2012; 862:75-87.), polymerase chain reaction (PCR) (See, e.g., Waters D L, Shapter F M. Methods Mol Biol. 2014; 1099:65-75.), DNA sequencing, allele specific oligonucleotide (ASO) probes (see, e.g. Stavljenic-Rukavina A, Sertic J, Salzer B, Dumic M, Radica A, Fumic K, Krajina A. Clin Chim Acta. 1993 Jul. 16; 216(1-2):191-8., and hybridization to DNA microarrays or beads. “APOE4 non-carrier” or “APOE negative subject” refers to a subject that does not carry an APOE4 allele. An APOE4 non carrier can be a carrier of other polymorphs of APOE in their genome including, but not limited to, APOE2 or APOE3.

“Cognitive performance” refers to measurable cognitive behavior or cognitive ability in a subject. There are various art-recognized tests for assessing cognitive performance in humans, for example and without limitation, the clinical global impression of change scale (CIBIC-plus scale); the Mini Mental State Exam (MMSE); the Neuropsychiatric Inventory (NPI); the Clinical Dementia Rating Scale (CDR); Clinical Dementia Rating-Sum of Boxes (CDR-SB); the Cambridge Neuropsychological Test Automated Battery (CANTAB); the Sandoz Clinical Assessment-Geriatric (SCAG), the Buschke Selective Reminding Test (Buschke and Fuld, 1974); the Verbal Paired Associates subtest; the Logical Memory subtest; the Visual Reproduction subtest of the Wechsler Memory Scale-Revised (WMS-R) (Wechsler, 1997); the Benton Visual Retention Test, or MATRICS consensus neuropsychological test battery which includes tests of working memory, speed of processing, attention, verbal learning, visual learning, reasoning and problem solving and social cognition. See Folstein et al., J Psychiatric Res 12: 189-98, (1975); Robbins et al., Dementia 5: 266-81, (1994); Rey, L'examen clinique en psychologie, (1964); Kluger et al., J Geriatr Psychiatry Neurol 12:168-79, (1999); Marquis et al., 2002 and Masur et al., 1994. Also see Buchanan, R. W., Keefe, R. S. E., Umbricht, D., Green, M. F., Laughren, T., and Marder, S. R. (2011), The FDA-NIMI-MATRICS guidelines for clinical trial design of cognitive-enhancing drugs: what do we know 5 years later? Schizophr. Bull. 37, 1209-1217. Another example of a cognitive test in humans is the explicit 3-alternative forced choice task. In this test, subjects are presented with color photographs of common objects consisting of a mix of three types of image pairs: similar pairs, identical pairs and unrelated foils. The second of the pair of similar objects is referred to as the “lure.” These image pairs are fully randomized and presented individually as a series of images. Subjects are instructed to make a judgment as to whether the objects seen are new, old or similar. A “similar” response to the presentation of a lure stimulus indicates successful memory retrieval by the subject. By contrast, calling the lure stimulus “old” or “new” indicates that correct memory retrieval did not occur.

In addition to assessing cognitive performance, the progression of cognitive impairment and dementia may be monitored by assessing surrogate changes in the brain of the subject. Surrogate changes include, without limitation, changes in regional brain volumes, perforant path degradation, and changes seen in brain function through resting state fMRI (R-fMRI), positron emission tomography (PET), single photon emission computed Tomography (SPECT), fluorodeoxyglucose positron emission tomography (FDG-PET), or any other imaging technique that allows one to measure brain function. Examples of regional brain volumes useful in monitoring the progression of cognitive impairment and dementia include reduction of hippocampal volume and reduction in volume or thickness of entorhinal cortex. These volumes may be measured in a subject by, for example, MRI. Aisen et al., Alzheimer's & Dementia 6:239-246 (2010). Perforant path degradation has been shown to be linked to age, as well as reduced cognitive performance. For example, older adults with more perforant path degradation tend to perform worse in hippocampus-dependent memory tests. Perforant path degradation may be monitored in subjects through ultrahigh-resolution diffusion tensor imaging (DTI). Yassa et al., PNAS 107:12687-12691 (2010). Resting-state fMRI (R-fMRI) involves imaging the brain during rest and recording large-amplitude spontaneous low-frequency (<0.1 Hz) fluctuations in the fMRI signal that are temporally correlated across functionally related areas. Seed-based functional connectivity, independent component analyses, and/or frequency-domain analyses of the signals are used to reveal functional connectivity between brain areas, particularly those areas whose connectivity increase or decrease with age, as well as the extent of cognitive impairment and/or dementia. FDG-PET uses the uptake of FDG as a measure of regional metabolic activity in the brain. Decline of FDG uptake in regions such as the posterior cingulated cortex, temporoparietal cortex, and prefrontal association cortex has been shown to relate to the extent of cognitive decline and dementia. Aisen et al., Alzheimer's & Dementia 6:239-246 (2010), Herholz et al., NeuroImage 17:302-316 (2002).

“Pharmaceutically acceptable salts” include, but are not limited to, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

“Pharmaceutically acceptable salt” includes both acid and base addition salts. “Pharmaceutically acceptable acid addition salt” may refer to those salts which retain the biological effectiveness and properties of the free bases, which are not biologically or otherwise undesirable, and which are formed with inorganic acids such as, but are not limited to, hydrohalic acids, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, hydroxyacetic acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

Conversely, said salt forms can be converted into the free forms by treatment with an appropriate base or acid.

### Compositions and Medicaments Useful in the Methods and of the Disclosure

Compositions and medicaments useful in the methods and uses of this disclosure are characterized by one or more of levetiracetam, brivaracetam or seletracetam, or pharmaceutically acceptable salts thereof. In some embodiments, the compositions and medicaments are characterized by levetiracetam or a pharmaceutically acceptable salt thereof.

Levetiracetam refers to the compound (2S)-2-(2-oxopyrrolidin-1-yl)butanamide (International Union of Pure and Applied Chemistry (IUPAC) name). Levetiracetam is a widely used antiepileptic drug. Levetiracetam binds to a specific site in the CNS: the synaptic vesicle protein 2A (SV2A) (See, e.g., Noyer et al. 1995; Fuks et al. 2003; Lynch et al. 2004; Gillard et al. 2006) and has further been shown to directly inhibit synaptic activity and neurotransmission by inhibiting presynaptic neurotransmitter release (Yang et al., 2007). Levetiracetam is sold as the FDA approved antiepileptic drug Keppra®. Typically, the therapeutically effective dose of levetiracetam (Keppra®) is in a range of 1000-3000 mg/day.

Levetiracetam is rapidly and almost completely absorbed after oral administration, and its bioavailability is not affected by food. Plasma half-life of levetiracetam is approximately 7±1 hour (expected to be 9-10 hours in elderly due to decreased renal function). Absorption is rapid, with peak plasma concentrations occurring about 1 hour following oral administration. Steady state can be achieved after 2 days of multiple twice-daily dosing.

A typical starting dose of levetiracetam in treating epilepsy in humans is 500 mg twice a day. The dosage is then increased until optimal efficacy, up to 3000 mg per day.

Brivaracetam refers to the compound (2S)-2-[(4R)-2-oxo-4-propylpyrrolidin-1-yl]butanamide (IUPAC name). It has anticonvulsant activity and binds to SV2A in the brain. It is approved under the name Briviact®. The typical starting dose is 50 mg orally twice per day, with a maintenance dose of 25-100 mg orally twice a day.

Seletracetam refers to the compound (2S)-2-[(4S)-4-(2,2-difluoroethenyl)-2-oxopyrrolidin-1-yl]butanamide (IUPAC name). It is an antiepileptic agent and binds to SV2A in the brain.

In some embodiments, the interval of administration of the levetiracetam, brivaracetam, or seletracetam, or the pharmaceutically acceptable salt thereof, or a pharmaceutical composition comprising any of the foregoing, is once every 12 hours (twice daily) or 24 hours (once daily). In some embodiments, once daily administration is used. Administration at less frequent intervals, such as once every 6 hours, may also be used.

In some embodiments, the levetiracetam, or a pharmaceutically acceptable salt thereof, itself or as part of a pharmaceutical composition, is administered at a daily dose of 70 mg to 140 mg, or 7 mg to 180 mg, or 25 mg to 180 mg, or 40 mg to 130 mg, or 140 to 300 mg, or 200 to 300 mg, or 140 to 200 mg, or 7 mg to 350 mg, 70 mg to 350 mg, 100 mg to 300 mg, or 125 mg to 250 mg. In some embodiments, the levetiracetam, or a pharmaceutically acceptable salt thereof, itself or as part of a pharmaceutical composition, is administered at a daily dose of 190 mg to 220 mg. In some embodiments, the levetiracetam, or a pharmaceutically acceptable salt thereof, itself or as part of a pharmaceutical composition, is administered at a daily dose of 190 mg to 240 mg. In some embodiments, the levetiracetam, or a pharmaceutically acceptable salt thereof, itself or as part of a pharmaceutical composition, is administered at a daily dose of 220 mg. In some embodiments, the levetiracetam, or a pharmaceutically acceptable salt thereof, itself or as part of a pharmaceutical composition, is administered at a daily dose of 190 mg.

In some embodiments of the methods of this disclosure, the levetiracetam, or a pharmaceutically acceptable salt thereof, or a pharmaceutical composition comprising levetiracetam, or a pharmaceutically acceptable salt thereof, is administered is in an oral form, extended-release form (e.g., a controlled release form, a prolonged release form, a sustained release form, a delayed release form, or a slow release form), or a single-unit-dosage form or a form for once-a-day administration. In some embodiments, the extended-release pharmaceutical composition is in the form of a tablet or capsule. In some embodiments, the levetiracetam, or the pharmaceutically acceptable salt thereof, or a pharmaceutical composition comprising levetiracetam, or a pharmaceutically acceptable salt thereof, is administered once or twice daily. In some embodiments, the levetiracetam or a pharmaceutically acceptable salt thereof, or a pharmaceutical composition comprising levetiracetam, or a pharmaceutically acceptable salt thereof, is administered once daily. In some embodiments, the levetiracetam or a pharmaceutically acceptable salt thereof, or a pharmaceutical composition comprising levetiracetam, or a pharmaceutically acceptable salt thereof, is administered once daily. In some embodiments, the administration is in an oral, single unit dosage, extended-release form. In some embodiments, the extended release pharmaceutical composition of levetiracetam or a pharmaceutically acceptable salt thereof is in a solid form.

In some embodiments of the disclosure, the brivaracetam, or the pharmaceutically acceptable salt thereof, itself or as part of a pharmaceutical composition, is administered at a daily dose of 7 to 15 mg, or 0.7 to 180 mg, or 2.5 to 180 mg, or 4.0 to 130 mg, or 14 to 30 mg. In other embodiments. the brivaracetam, or the pharmaceutically acceptable salt thereof, itself or as part of a pharmaceutical composition, is administered at a daily dose of 0.7-50 mg, 0.7-75 mg, 0.7-100 mg, 0.7-150 mg, 0.7-180 mg, 1.8-50 mg, 1.8-75 mg, 1.8-100 mg, 1.8-150 mg, 1.8-180 mg, 3.5-50 mg, 3.5-75 mg, 3.5-100 mg, 3.5-150 mg, 3.5-180 mg, 5-50 mg, 5-75 mg, 5-100 mg, 5-150 mg, 5-180 mg, 7-50 mg, 7-75 mg, 7-100 mg, 7-150 mg, 7-180 mg, 15-50 mg, 15-75 mg, 15-100 mg, 15-150 mg, 15-180 mg, 35-50 mg, 35-75 mg, 35-100 mg, 35-150 mg, 35-180 mg.

In some embodiments, the brivaracetam, or a pharmaceutically acceptable salt thereof, or a pharmaceutical composition comprising brivaracetam, or a pharmaceutically acceptable salt thereof, is administered is in an oral form, extended release form (e.g., a controlled release form, a prolonged release form, a sustained release form, a delayed release form, or a slow release form), or a single-unit-dosage form or a form for once-a-day administration. In some embodiments, the extended-release pharmaceutical composition is in the form of a tablet or capsule. In some embodiments, the brivaracetam, or the pharmaceutically acceptable salt thereof, or a pharmaceutical composition comprising brivaracetam, or a pharmaceutically acceptable salt thereof, is administered once or twice daily. In some embodiments, the brivaracetam or pharmaceutical composition thereof is administered once daily in single use, extended-release form. In some embodiments, the extended-release pharmaceutical composition is in a solid form. In some embodiments, the extended-release pharmaceutical composition is in the form of a tablet or capsule. In some embodiments, the brivaracetam or a pharmaceutically acceptable salt thereof, or a pharmaceutical composition comprising brivaracetam, or a pharmaceutically acceptable salt thereof, is administered once daily. In some embodiments, the brivaracetam or a pharmaceutically acceptable salt thereof, or a pharmaceutical composition comprising brivaracetam, or a pharmaceutically acceptable salt thereof, is administered once daily. In some embodiments, the administration is in an oral, single unit dosage, extended-release form. In some embodiments, the extended release pharmaceutical composition of brivaracetam or a pharmaceutically acceptable salt thereof is in a solid form.

In some embodiments, the seletracetam, or a pharmaceutically acceptable salt thereof, itself or as part of a pharmaceutical composition, is administered at a daily dose of 70 mg to 140 mg, or 7 mg to 180 mg, or 25 mg to 180 mg, or 40 mg to 130 mg, or 140 to 300 mg, or 200 to 300 mg, or 140 to 200 mg, or 7 mg to 350 mg, 70 mg to 350 mg, 100 mg to 300 mg, or 125 mg to 250 mg. In some embodiments, the seletracetam, or a pharmaceutically acceptable salt thereof, itself or as part of a pharmaceutical composition, is administered at a daily dose of 190 mg to 220 mg. In some embodiments, the seletracetam, or a pharmaceutically acceptable salt thereof, itself or as part of a pharmaceutical composition, is administered at a daily dose of 190 mg to 240 mg. In some embodiments, the seletracetam, or a pharmaceutically acceptable salt thereof, itself or as part of a pharmaceutical composition, is administered at a daily dose of 220 mg. In some embodiments, the seletracetam, or a pharmaceutically acceptable salt thereof, itself or as part of a pharmaceutical composition, is administered at a daily dose of 190 mg.

In some embodiments of the methods of this disclosure, the seletracetam, or a pharmaceutically acceptable salt thereof, or a pharmaceutical composition comprising seletracetam, or a pharmaceutically acceptable salt thereof, is administered is in an oral form, extended-release form (e.g., a controlled release form, a prolonged release form, a sustained release form, a delayed release form, or a slow release form), or a single-unit-dosage form or a form for once-a-day administration. In some embodiments, the extended-release pharmaceutical composition is in the form of a tablet or capsule. In some embodiments, the seletracetam, or the pharmaceutically acceptable salt thereof, or a pharmaceutical composition comprising seletracetam, or a pharmaceutically acceptable salt thereof, is administered once or twice daily. In some embodiments, the seletracetam or a pharmaceutically acceptable salt thereof, or a pharmaceutical composition comprising seletracetam, or a pharmaceutically acceptable salt thereof, is administered once daily. In some embodiments, the seletracetam or a pharmaceutically acceptable salt thereof, or a pharmaceutical composition comprising seletracetam, or a pharmaceutically acceptable salt thereof, is administered once daily. In some embodiments, the administration is in an oral, single unit dosage, extended-release form. In some embodiments, the extended release pharmaceutical composition of seletracetam or a pharmaceutically acceptable salt thereof is in a solid form.

In some embodiments of the methods and uses of this disclosure, the daily dose of the levetiracetam or pharmaceutically acceptable salt thereof in the pharmaceutical composition is 220 mg and the pharmaceutical composition further comprises 280 mg-350 mg of hydroxypropyl methylcellulose, 1.2 mg-1.4 mg of colloidal silicon dioxide, 92.8 mg-119.2 mg of silicified microcrystalline cellulose, and 6.0 mg-6.7 mg of magnesium stearate. In other embodiments, the daily dose of the levetiracetam or pharmaceutically acceptable salt thereof in the pharmaceutical composition is 220 mg and the pharmaceutical composition further comprises 280 mg of hydroxypropyl methylcellulose, 1.2 mg of colloidal silicon dioxide, 92.8 mg of silicified microcrystalline cellulose, and 6.0 mg of magnesium stearate. In other embodiments, the daily dose of the levetiracetam or pharmaceutically acceptable salt thereof in the pharmaceutical composition is 220 mg and the pharmaceutical composition further comprises 347.5 mg of hydroxypropyl methylcellulose, 1.4 mg of colloidal silicon dioxide, 119.2 mg of silicified microcrystalline cellulose, and 6.7 mg of magnesium stearate. In some embodiments, the hydroxypropyl methylcellulose is hypromellose 2208. In some embodiments, the silicified microcrystalline cellulose is silicified microcrystalline cellulose SMCC 90. In some embodiments, the composition is in unit dosage form for once daily administration. In some embodiments, the composition is in oral, extended-release form. In some embodiments, the extended-release pharmaceutical composition is in a solid form. In some embodiments, the extended-release pharmaceutical composition is in the form of a tablet or capsule. In some embodiments the oral, extended release form is 220 mg in unit dosage form for once daily administration.

In some embodiments of the methods and uses of this disclosure, the daily dose of the levetiracetam in the pharmaceutical composition is 220 mg and the pharmaceutical composition further comprises 280 mg-350 mg of hydroxypropyl methylcellulose, 1.2 mg-1.4 mg of colloidal silicon dioxide, 92.8 mg-119.2 mg of silicified microcrystalline cellulose, and 6.0 mg-6.7 mg of magnesium stearate. In other embodiments, the daily dose of the levetiracetam in the pharmaceutical composition is 220 mg and the pharmaceutical composition further comprises 280 mg of hydroxypropyl methylcellulose, 1.2 mg of colloidal silicon dioxide, 92.8 mg of silicified microcrystalline cellulose, and 6.0 mg of magnesium stearate. In other embodiments, the daily dose of the levetiracetam in the pharmaceutical composition is 220 mg and the pharmaceutical composition further comprises 347.5 mg of hydroxypropyl methylcellulose, 1.4 mg of colloidal silicon dioxide, 119.2 mg of silicified microcrystalline cellulose, and 6.7 mg of magnesium stearate. In some embodiments, the hydroxypropyl methylcellulose is hypromellose 2208.

In some embodiments of the methods and uses of this disclosure the daily dose of the levetiracetam or pharmaceutically acceptable salt thereof in the pharmaceutical composition is 190 mg and the pharmaceutical composition further comprises 300 mg of hydroxypropyl methylcellulose, 1.2 mg of colloidal silicon dioxide, 102.8 mg of silicified microcrystalline cellulose or anhydrous dicalcium phosphate, and 6 mg of magnesium stearate. In some embodiments of the methods and uses of this disclosure the daily dose of the levetiracetam in the pharmaceutical composition is 190 mg and the pharmaceutical composition further comprises 300 mg of hydroxypropyl methylcellulose, 1.2 mg of colloidal silicon dioxide, 102.8 mg of silicified microcrystalline cellulose or anhydrous dicalcium phosphate, and 6 mg of magnesium stearate. In some embodiments, the hydroxypropyl methylcellulose is hypromellose 2208. In some embodiments, the hydroxypropyl methylcellulose is hypromellose 2208. In some embodiments, the silicified microcrystalline cellulose is silicified microcrystalline cellulose SMCC 90. In some embodiments, the composition is in unit dosage form for once daily administration. In some embodiments, the composition is in oral, extended-release form. In some embodiments, the extended-release pharmaceutical composition is in a solid form. In some embodiments, the extended-release pharmaceutical composition is in the form of a tablet or capsule. In some embodiments the oral, extended release form is 190 mg in unit dosage form for once daily administration.

In some embodiments, the pharmaceutical composition comprising the daily dose of the levetiracetam or pharmaceutically acceptable salt thereof is in extended release form and provides a steady state plasma concentration of levetiracetam in the subject of between 1.9 μg/mL and 4.4 μg/mL within 3 hours after administration and extending for at least 8 hours of a 24-hour period after said administration. In some embodiments, the pharmaceutical composition comprising the daily dose of the levetiracetam or pharmaceutically acceptable salt thereof is in extended release form and provides a steady state plasma concentration of levetiracetam in the subject of between 1.9 μg/mL and 4.4 μg/mL within 2 hours after said administration and extending for at least 13 hours of a 24-hour period after administration. In some embodiments, the pharmaceutical composition comprising the daily dose of the levetiracetam or pharmaceutically acceptable salt thereof is in extended release form and provides a steady state plasma concentration of levetiracetam in the subject of between 1.9 μg/mL and 4.4 μg/mL within 1 hour after said administration and extending for at least 13 hours of a 24-hour period after administration. In some embodiments, the pharmaceutical composition comprising the daily dose of the levetiracetam is in extended release form and provides a steady state plasma concentration of levetiracetam in the subject of between 1.9 μg/mL and 4.4 μg/mL within 3 hours after administration and extending for at least 8 hours of a 24-hour period after said administration. In some embodiments, the pharmaceutical composition comprising the daily dose of the levetiracetam is in extended release form and provides a steady state plasma concentration of levetiracetam in the subject of between 1.9 μg/mL and 4.4 μg/mL within 2 hours after said administration and extending for at least 13 hours of a 24-hour period after administration. In some embodiments, the pharmaceutical composition comprising the daily dose of the levetiracetam is in extended release form and provides a steady state plasma concentration of levetiracetam in the subject of between 1.9 μg/mL and 4.4 μg/mL within 1 hour after said administration and extending for at least 13 hours of a 24-hour period after administration. In other embodiments, the pharmaceutical composition provides said steady state plasma concentration of levetiracetam in the subject of between 1.9 μg/mL and 4.4 μg/mL within 1 hour after administration and extending for at least 13 to 16 hours of a 24-hour period after said administration. See, e.g. WO2016191288.

In some embodiments, the pharmaceutical composition comprising the daily dose of the levetiracetam or pharmaceutically acceptable salt thereof is formulated in one or more of an oral form, an extended release form or a single-unit-dosage-form or for once-a-day administration. In some embodiments, the pharmaceutical composition comprising the daily dose of the levetiracetam is formulated in one or more of an oral form, an extended release form or a single-unit-dosage-form or for once-a-day administration. In other embodiments, the extended release form is a controlled release form, a prolonged release form, a sustained release form, a delayed release form, or a slow release form. In some embodiments, the extended release pharmaceutical composition of levetiracetam or a pharmaceutically acceptable salt thereof is in a solid form. In some embodiments, the extended release pharmaceutical composition of levetiracetam or a pharmaceutically acceptable salt thereof is in the form of a tablet or capsule.

Table 1 provides a description of three extended-release oral formulations of levetiracetam (190 mg Tablets A, B, and C). In some embodiments, the pharmaceutical composition useful in the methods and uses of this disclosure is selected form the group of the formulations in Table 1. In one embodiment of the methods and uses of this disclosure, the pharmaceutical composition is the 190 mg Tablet A formulation.

TABLE 1
A process for making extended-release compositions comprising 190 mg of
levetiracetam

| | | Tablet A | Tablet B | Tablet C |
|---|---|---|---|---|
| Ingredient | Functionality | (Mg/Tablet) | (Mg/Tablet) | (Mg/Tablet) |
| Levetiracetam Base | API | 190.0 | 190.0 | 190.0 |
| Hypromellose | Matrix | 300.0 | — | — |
| (Methocel ™ K15M CR) | Former | | | |
| Hypromellose | Matrix | — | 300.0 | 300.0 |
| (Methocel ™ K100M | Former | | | |
| Premium CR) | | | | |
| Colloidal Silicon | Glidant | 1.2 | 1.2 | 1.2 |
| Dioxide | | | | |
| Silicified | Diluent | 102.8 | 102.8 | — |
| Microcrystalline | | | | |
| Cellulose | | | | |
| ProSolv ™ HD90 | | | | |
| Encompress, Anhydrous | Diluent | — | — | 102.8 |
| dicalcium phosphate | | | | |
| Magnesium Stearate | Lubricant | 6.0 | 6.0 | 6.0 |

| | | | |
|---|---|---|---|
| Total | 600 | 600 | 600 |

Table 2 provides a description of two extended-release formulations of levetiracetam (220 mg Tablets D and E). In some embodiments of the methods and uses of this disclosure, the pharmaceutical composition is selected from the group of the formulations in Table 2. In one embodiment, the pharmaceutical composition is the 220 mg Tablet D formulation.

TABLE 2
A process for making extended-release compositions
comprising 220 mg of levetiracetam

| | | Tablet D | Tablet E |
|---|---|---|---|
| Ingredient | Functionality | (Mg/Tablet) | (Mg/Tablet) |
| Levetiracetam | API | 220.0 | 220.0 |
| Hypromellose (Methocel ™ | Matrix Former | 280.0 | 347.5 |
| K15M CR) | | | |
| Colloidal Silicon Dioxide | Glidant | 1.2 | 1.4 |
| Silicified Microcrystalline | Diluent | 92.8 | 119.2 |
| Cellulose | | | |
| ProSolv ™ HD90 | | | |
| Magnesium Stearate | Lubricant | 6.0 | 6.7 |

| | | |
|---|---|---|
| Total | 600 | 695 |

In addition to oral delivery, the pharmaceutical compositions and medicaments useful in the methods and uses of this disclosure may also be formulated for respiratory delivery (pulmonary and nasal delivery). In such form, they may be delivered by devices and in forms that include but are not limited to a variety of pressurized metered dose inhalers, dry powder inhalers, nebulizers, aqueous mist inhalers, drops, solutions, suspensions, sprays, powders, gels, ointments, and specialized systems such as liposomes and microspheres (see e.g. Owens D R, Zinman B, Bolli G. Alternative routes of insulin delivery. Diabet Med. 2003 November; 20(11):886-98 and Martini G, Ciani L. Electron spin resonance spectroscopy in drug delivery. Phys Chem Phys. 2009).

Pharmaceutical compositions and medicaments useful in the methods and uses of this disclosure may also be formulated for transdermal delivery using formats, including but are not limited to colloids, patches, and microemulsions.

The pharmaceutical compositions and medicaments useful in the methods and uses of this disclosure may also comprise adjuvants, such as preservatives, wetting agents, emulsifying agents and dispersing agents. Prevention of the action of microorganisms may be afforded by the inclusion of various antibacterial and antifungal agents, for example, paraben, chlorobutanol, phenol sorbic acid, and the like. It may also be desirable to include isotonic agents, such as sugars, sodium chloride, and the like into the pharmaceutical composition.

The pharmaceutical compositions and medicaments useful in the methods and uses of this disclosure can be prepared by methods well known in the art of pharmacy, see, e.g., Goodman and Gilman's The Pharmacological Basis of Therapeutics. 10th Edition Edited by J. G. Hardman, L. E. Limbird, and A. G. Gilman. McGraw Hill, New York. 2001; Ansel et al., Pharmaceutical Calculations, The Pharmacist's Handbook 2004, Lippincott Williams & Wilkins; Stoklosa et al., 2001 Pharmaceutical Calculations 11th edition (9780781731720)—Textbooks.com; and Bustamante et al., “A Modification of the Extended Hildebrand Approach to Predict the Solubility of Structurally Related Drugs in Solvent Mixtures,” Journal of Pharmacy and Pharmacology, 45:253-257 (1993).

### EXAMPLES

### Example 1: Compositions of Levetiracetam

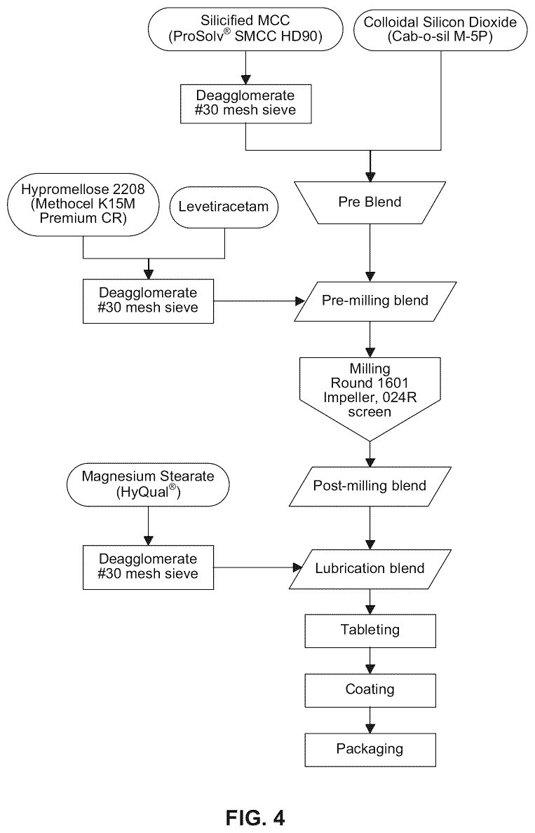

Compositions comprising levetiracetam can be made through the process exemplified in the flow diagram of FIG. 4. In brief, Silicified Microcrystalline Cellulose ProSolv™ SMCC HD90 (or Encompress, Anhydrous dicalcium phosphate) is sifted through deagglomerate #30 U.S. mesh sieve, and then blended with Colloidal Silicon Dioxide (16 qt V-shell blender; 75 rev±5 rev). The blended sample then goes through Round 1601 Impeller (2A024R screen). 220 mg of levetiracetam and hypromellose 2208 (Methocel™ K15M Premium CR) (or Methocel™ K100M Premium CR) are also sifted through deagglomerate #30 U.S. mesh sieve, and then blended in a 1 ft³ Slant Cone Blender (250 rev±5 rev) with the ground Silicified Microcrystalline Cellulose ProSolv™ HD90 and Colloidal Silicon Dioxide. This blended sample then goes through Round 1601 Impeller (2A024R screen) and then is blended in a 1 ft³ Slant Cone Blender (125 rev±5 rev) with sieved Magnesium Stearate (HyQual®) (sieved through deagglomerate #30 U.S. mesh sieve). The blended samples are compressed into tablets. Optionally, the tablets are further film coated with a hypromellose-based (HPMC-based) coating, such as Opadry® complete film coating system. A similar process can be used for compositions comprising 190 mg of levetiracetam.

### Example 2: Evaluation of Levetiracetam Blood Plasma Levels

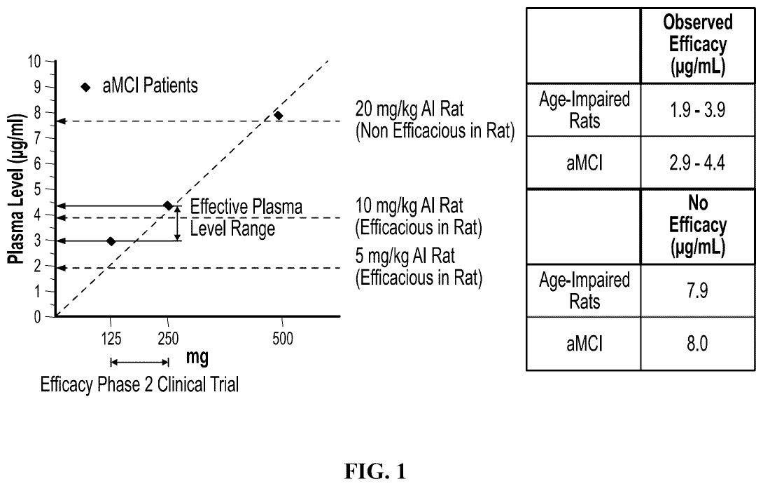

Steady state levetiracetam plasma concentration ranges effective for treating cognitive impairment were previously established using the aMCI clinical trials and aged rat studies disclosed in WO2016191288, which is incorporated by reference herein in its entirety. In one embodiment, the concentration range of the levetiracetam is between 2.9 and 4.4 μg/mL. In another embodiment, the concentration range of the levetiracetam is between 1.9 and 4.4 μg/mL. In another aspect, the effective plasma concentration is between 1.9 and 3.9 μg/mL. See FIG. 1.

This example further describes a two-group, single-dose, two-period, two-way crossover, food-effect study of two extended release levetiracetam formulations, i.e., the 190 mg Tablet A of Table 1 and the 220 mg Tablet D of Table 2.

### Study Design

This is an open label, randomized, two-group, single-dose, two-period crossover, food-effect study. Fifty-six (56) healthy subjects are enrolled. Subjects who successfully complete the screening process check into the research center the evening before first dose. Subjects who continue to meet inclusion/exclusion criteria the morning of dose are assigned a subject number, based on the order in which they successfully complete the required screening process and procedures. Dosing days are separated by a washout period of at least 7 days. Subjects are randomly assigned to one of two groups:

Group 1: Subjects (n = 28) received extended-release
Tablet A of Table 1 (190 mg).

| | |
|---|---|
| Treatment | Tablet A |
| A: | Dose = 1 × 190 mg tablet, orally |
| | administered under fasted conditions |
| Treatment | Tablet A |
| B: | Dose = 1 × 190 mg tablet, orally |
| | administered under fed conditions |

Group 2: Subjects (n = 28) received extended-release
Tablet D of Table 2 (220 mg).

| | |
|---|---|
| Treatment A: | Tablet D |
| | Dose = 1 × 220 mg tablet, orally |
| | administered under fasted conditions |
| Treatment B: | Tablet D |
| | Dose = 1 × 220 mg tablet, orally |
| | administered under fed conditions |

### Clinical Procedures Summary

During each study period, 6 mL blood samples are obtained prior to each dosing and following each dose at selected times through 24 hours post-dose. A total of 34 pharmacokinetic blood samples are to be collected from each subject, 17 samples in each study period. In addition, blood is drawn and urine is collected for clinical laboratory testing at screening and study exit.

In each study period, subjects are admitted to the study unit in the evening prior to the scheduled dose. Subjects are confined to the research center during each study period until completion of the 24-hour blood collection and other study procedures.

### Procedures for Collecting Samples for Pharmacokinetic Analysis

Blood samples (1×6 mL) are collected in vacutainer tubes containing K2-EDTA as a preservative at pre-dose (0) and at 1.0, 2.0, 3.0, 4.0, 4.5, 5.0, 5.5, 6.0, 6.5, 7.0, 8.0, 9.0, 10, 12, 18, and 24 hours after dosing.

### Bioanalytical Summary

Plasma samples are analyzed for levetiracetam using a validated LC MS procedure. The method is validated for a range of 0.0500 to 30.0 μg/mL for levetiracetam, based on the analysis of 0.200 mL of human EDTA plasma. Data are stored in Watson Laboratory Information Management System (LIMS; Version 7.2.0.03, Thermo Fisher Scientific).

### Pharmacokinetic Analysis

Data are analyzed by noncompartmental methods in WinNonlin. Concentration time data that are below the limit of quantification (BLQ) are treated as zero in the data summarization and descriptive statistics. In the pharmacokinetic analysis, BLQ concentrations are treated as zero from time-zero up to the time at which the first quantifiable concentration is observed; embedded and/or terminal BLQ concentrations are treated as “missing”. Actual sample times are used for all pharmacokinetic and statistical analyses.

The following pharmacokinetic parameters are calculated: peak concentration in plasma (Cₘₐₓ), time to peak concentration (Tₘₐₓ), elimination rate constant (λz), terminal half-life (T_{1/2}), area under the concentration-time curve from time-zero to the time of the last quantifiable concentration (AUCₗₐₛₜ), and area under the plasma concentration time curve from time-zero extrapolated to infinity (AUC_{inf}). Additionally, Cₘₐₓ, AUCₗₐₛₜ, and AUC_{inf} are dose-normalized.

### Steady State Modeling

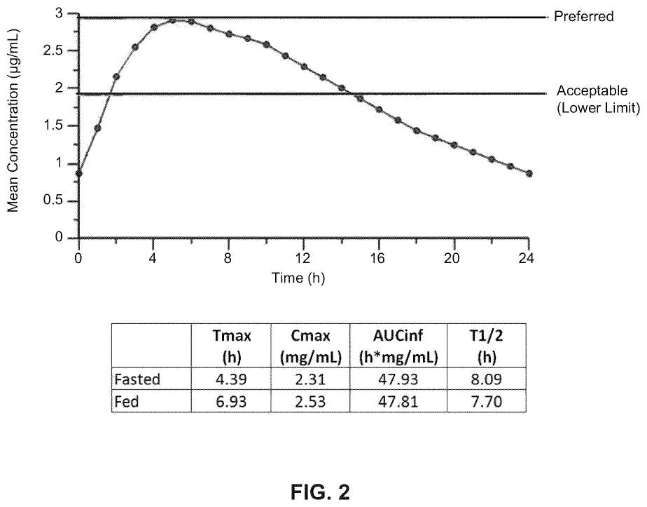

According to the steady state modeling of PK profile for the 190 mg Tablet A, the plasma concentrations of the levetiracetam were between 1.9 and 4.4 μg/mL for substantial parts of the 24-hour period after administration. See FIG. 2.

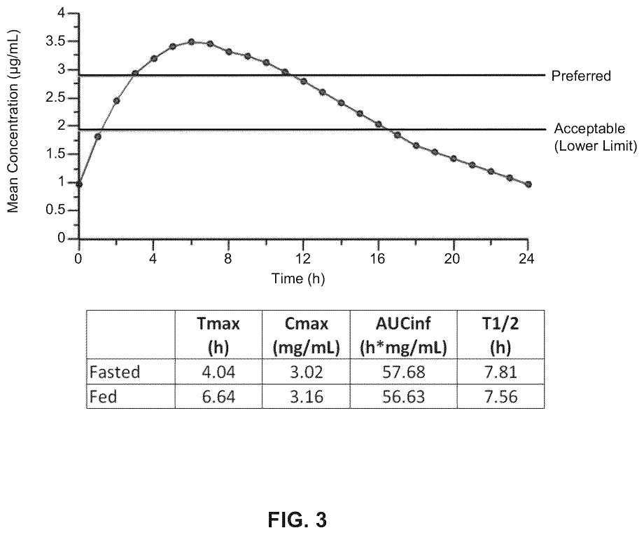

According to the steady state modeling of PK profile for the 220 mg Tablet D, the plasma concentrations of the levetiracetam were between 2.9 and 4.4 μg/mL for substantial parts of the 24-hour period after administration. See FIG. 3.

### Example 3: Evaluation of 220 mg Levetiracetam Extended-Release Tablet for Slowing the Progression of Mild Cognitive Impairment (MCI) Due to Alzheimer's Disease (AD)

This example describes a phase IIb multicenter, randomized, double-blind, placebo-controlled study evaluating the efficacy and safety of a low-dose levetiracetam, 220 mg, extended-release tablet on slowing progression of MCI due to Alzheimer's disease (prodromal Alzheimer's disease), the results being evaluated using the well-recognized and accepted Clinical Dementia Rating-Sum of Boxes (CDR-SB) scores.

### Subjects

Subjects for this study were between 55 and 85 years old (inclusive) in good general health, willing and able to consent and to participate for the duration of the study, having an eighth-grade education or good work history sufficient to exclude mental retardation, having visual and auditory acuity adequate for neuropsychological testing, and having proficient fluency of the native local language to participate in all the neuropsychological test assessments. The clinical assessments of 63 participants were used for the analyses.

### APOE4 Carrier Status Determination

Blood samples were taken from each subject to determine APOE4 carrier status. Genomic DNA was prepared from the blood samples according to standard procedures. Quality of the genomic DNA samples were quantified using Optical Density and DNA purity was calculated. Using extracted genomic DNA as a template, locus specific DNA fragments were amplified by polymerase chain reaction (PCR). Purified PCR products were used as templates for sequencing reactions. According to the chain terminating methodology of Sanger et al (Sanger F, Nicklen S, Coulson A R. DNA sequencing with chain-terminating inhibitors. Proc Natl Acad Sci USA. 1977 December; 74(12):5463-7), analysis of DNA sequence was based on the termination of a growing DNA strand due to incorporation of a dye labeled ddNTP by the DNA polymerase. The resulting dye-labeled product was detected using an automated sequencing platform. Data was analyzed using the software SEQPATIENT from JSI-medical Systems®.

### Study Design

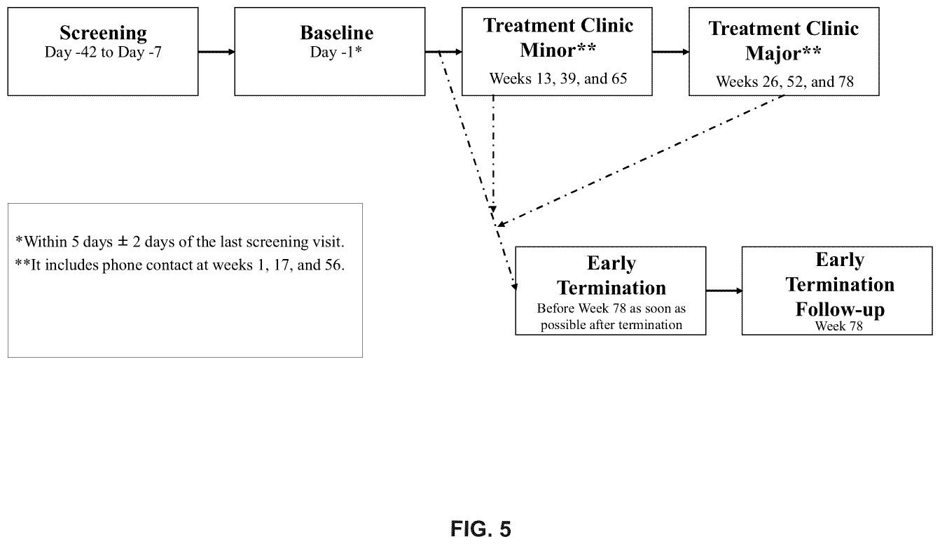

The study included up to a 5-week screening period consisting of 3 visits and a 78-week treatment period consisting of 10 visits (1 baseline visit, 3 telephone call visits, and 6 clinic visits). See FIG. 5. The primary endpoint of the study was the analysis of the change in CDR-SB score from baseline to 78 weeks. Secondary endpoints included the analysis of the change in Functional Activities Questionnaire (FAQ) score from baseline to 78 weeks. Subjects received a low-dose levetiracetam, 220 mg, extended-release tablet or placebo in the morning in identically appearing extended release, one unit oral dosage forms.

### Clinical Dementia Rating-Sum of Boxes (CDR-SB)

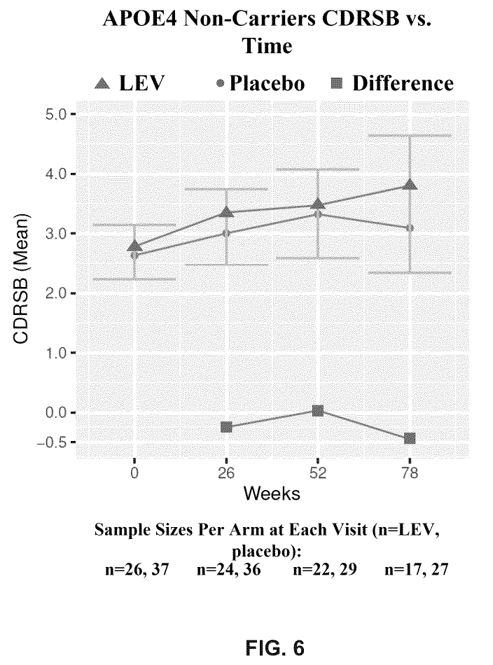

CDR-SB testing was performed using standard methods. Briefly, subjects were tested using the following six categories or “boxes”: Memory, Orientation, Judgment and Problem Solving, Community Affairs, Home and Hobbies, and Personal Care. CDR-SB scores were calculated by adding the box scores. The total score ranges from 0 to 18 with a higher scores indicating more impairment). Absolute values and changes from baseline in CDR-SB were summarized using descriptive statistics and presented by APOE4 non-carrier status. Table 3 summarizes the statistical analysis wherein each cell contains a point estimate followed by a 95% Confidence Interval (CI). FIG. 6 demonstrates the graphical representation of CDR-SB scores at baseline, 26 weeks, 52 weeks, and 78 weeks after levetiracetam (LEV) or placebo treatment. Briefly, APOE4 non-carriers demonstrated a surprising and unexpected 36.8% decrease of CDR-SB Change Score after 78 weeks of treatment with low-dose levetiracetam, 220 mg, extended-release tablet compared with placebo treated APOE4 non-carriers with the largest effect shown between 52 and 78 weeks of treatment.

TABLE 3
CDR-SB Change Score as Outcome
Estimated Treatment Effect and Mean

| | APOE4 Non-Carriers (n = 63) |
|---|---|
| LEV change score | 0.96 (CI: 0.08, 2.01) |
| Placebo change score | 1.52 (CI: 0.85, 2.62) |
| Treatment Effect (Difference) | −0.56 (CI: −1.90, 0.47) |

Exemplary methods and materials are described, although methods and materials similar or equivalent to those described herein can also be used in the practice or testing of the various aspects and embodiments. The materials, methods, and examples are illustrative only and not intended to be limiting.

The analysis of the primary efficacy endpoint revealed that for the entire study population, the mean CDR-SB change score at Week 78 was 1.12 for the levetiracetam group and 1.22 for the placebo group, with an estimated average treatment effect of −0.10 (p=0.71). For the APOE-4 carrier subgroup, the mean CDR-SB change score at Week 78 was 0.95 for the levetiracetam group (n=54) and 1.05 for the placebo group (n=45), with an estimated average treatment effect of −0.10. However, for the APOE-4 non-carrier subgroup, the mean change from baseline at Week 78 was 0.68 for the levetiracetam group (n=26) and 1.13 for the placebo group (n=37), with an estimated average treatment effect of −0.45.

### Secondary Outcomes—Functional Activities Questionnaire (FAQ) Scores

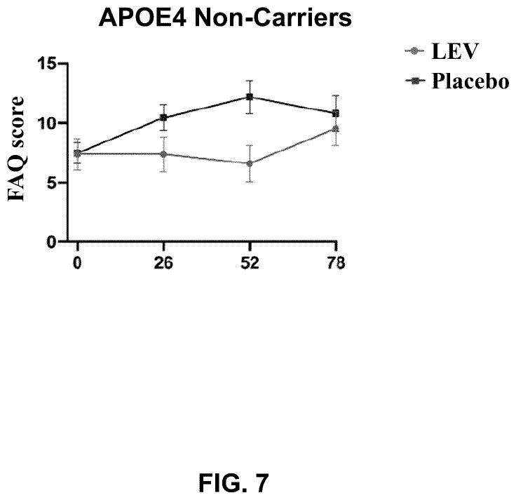

FAQ scores were obtained to measure the functional ability of individuals over time. Briefly, FAQ measures instrumental activities of daily life, such as preparing meals and balancing a check book as these functional changes are found earlier in the dementia process compared to more basic activities. The Questionnaire comprises 10 questions which are answered using a 0-3 scoring system for each question with 0 being normal and 3 being dependent on someone else to complete the task. Accordingly, the sum score ranges from 0-30 with higher scores associated with greater impairment. FIG. 7 demonstrates the graphical representation of FAQ scores at baseline, 26 weeks, 52 weeks, and 78 weeks after levetiracetam (LEV) or placebo treatment. Briefly, APOE4 non-carriers demonstrated a surprising and unexpected decrease of FAQ Score after 78 weeks of treatment with low-dose levetiracetam, 220 mg, extended-release tablet compared with placebo treated APOE4 non-carriers with the largest effect shown between 26 and 52 weeks of treatment.

Exemplary methods and materials are described, although methods and materials similar or equivalent to those described herein can also be used in the practice or testing of the various aspects and embodiments. The materials, methods, and examples are illustrative only and not intended to be limiting.

The analysis of the secondary efficacy endpoint revealed that for the entire study population the estimated mean FAQ score from baseline at 78 weeks is 3.82 (95% CI: 2.10, 5.17) for the levetiracetam group and 3.81 (95% CI: 2.38, 5.59) for the placebo group. For the APOE-4 carrier subgroup, the mean FAQ total score change from baseline to week 78 was 5 for the levetiracetam group (n=54) and 3.3 for the placebo group (n=45). However, for the APOE-4 non-carrier subgroup, the mean FAQ total score change from baseline to week 78 was 1.8 for the levetiracetam group (n=26) and 4.1 for the placebo group (n=37).

### Volumetric MRI of Medial Temporal Lobe Subregions

To examine whether treatment with levetiracetam showed an effect on cortical brain atrophy in the APOE-4 non-carriers (same subjects and groups as above) in addition to the effect observed on the CDR-SB score, an analysis of cortical volume as a function of treatment status was conducted. Brain regions of interest were selected prior to the trial apriori based on the location of post-mortem tau accumulation in the early stage of AD (Braak et al., Acta Neuropathologica, 112(4): 389-404 (2006); Braak & Braak, Acta Neuropathologica, 80(5): 479-486 (1990)) and robust atrophy in entorhinal cortex volume in the AD. For this purpose, the Automated Segmentation of Hippocampal Subfields (ASHS) software (Xie et al., Hum Brain Mapp. 40:3431-3451 (2019)) was used. Regions provided by this software include the left and right entorhinal cortex (ERC), Brodmann area 35 (BA35), which largely overlaps with TEC (Braak et al., Acta Neuropathologica, 112(4): 389-404 (2006); Braak & Braak, Acta Neuropathologica, 80(5): 479-486 (1990))) and includes a portion of the perirhinal cortex and BA36, which predominantly encompasses the perirhinal cortex (Xie et al., 2019).

High-resolution T1-weighted MRI images of the subjects' brain were collected at baseline and 78 weeks using an MRI scanner.

### Automatic Segmentation Using ASHS

Automatic segmentation was performed using the T1-weighted MRI images using the ASHS-T1 open-source software and see, also Xie et al., Hum Brain Mapp. 40:3431-3451 (2019).

The T1-weighted MRI images were analyzed using the ASHS-T1 open-source software and see, also Xie et al., Hum Brain Mapp. 40:3431-3451 (2019). Volumetric and thickness measurements of the ERC and BA35 were extracted for each subject. The quality of all the automatic segmentation generated by ASHS was visually checked. The pipeline labeled the baseline and week 78 T1-weighted MRI scans of all the subjects.

All statistical analyses were two-tailed with significance levels of p=0.05.

### Results

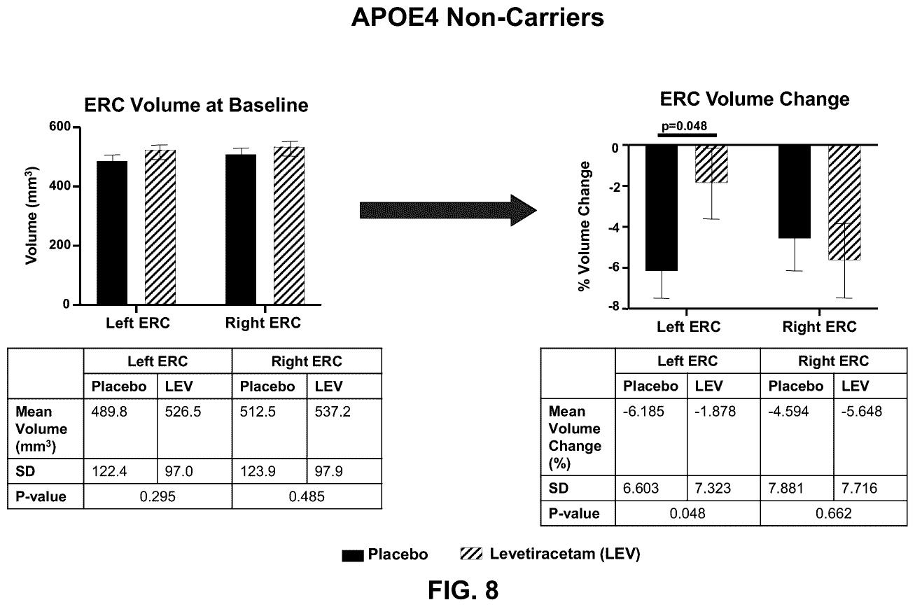

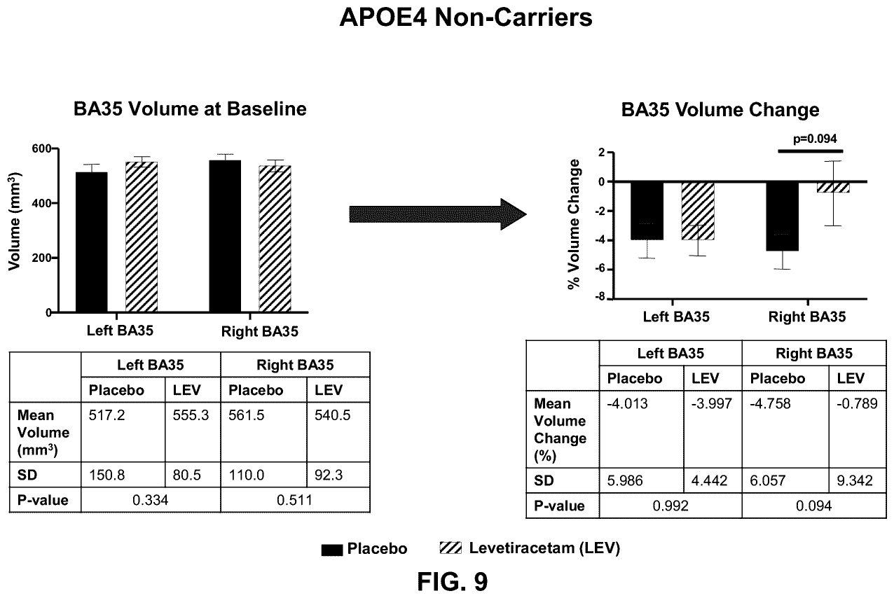

For the APOE-4 non-carrier subjects, the ERC at baseline did not differ in volume between those that were assigned to the placebo group (n=37) or the levetiracetam treatment group (n=26). At the week 78 end of trial visit, the left ERC volume change showed a significant reduction of atrophy in the levetiracetam treated group compared to the placebo group (LEV treated group: −1.878% mean volume change in volume, placebo group: −6.185% mean volume change in volume (p=0.048)) (FIG. 8). Volume at baseline in the BA35 in the APOE-4 non-carriers also did not differ between the groups at baseline. At the week 78 end of trial visit, the right BA35 volume change showed some reduction of atrophy in the levetiracetam treated group compared to the placebo group (LEV treated group: −0.789% mean volume change in volume, placebo group: −4.758% mean volume change in volume (p=0.094)) (FIG. 9).

1. A method of preventing or slowing the progression of cognitive impairment or preventing the development or reducing the rate of cognitive decline in an APOE4 non-carrier subject, the method comprising administering to the subject one or more of levetiracetam, brivaracetam or seletracetam, or a pharmaceutically acceptable salt thereof, wherein the levetiracetam, brivaracetam or seletracetam are administered at a daily dose of 0.7-350 mg, or comprising administering to the subject a pharmaceutical composition comprising the daily dose of the levetiracetam, brivaracetam or seletracetam, or a pharmaceutically acceptable salt thereof, and a pharmaceutically acceptable carrier.

2. The method of claim 1, wherein the APOE4 non-carrier subjects carry a genetic risk factor for the development of cognitive impairment which is not APOE4.

3. The method of claim 1, wherein the subject presents or displays with cognitive performance below the normal range for their age.

4. The method of claim 1, wherein the subject presents or displays with volumetric atrophy of a subregion of the medial temporal lobe.

5. The method of claim 4, wherein the subregion of the medial temporal lobe is the entorhinal cortex (ERC) or the transentorhinal cortex (BA35).

6.-8. (canceled)

9. The method of claim 1, wherein the subject presents with or displays pre-mild cognitive impairment, mild cognitive impairment, amnestic mild cognitive impairment (aMCI), mild cognitive impairment due to Alzheimer's disease (AD) or mild cognitive impairment due to prodromal AD.

10.-17. (canceled)

18. The method of claim 1 wherein the pharmaceutical composition is formulated in one or more of an oral form, an extended-release form, a single-unit-dosage form or a once-a-day form.

19.-20. (canceled)

21. The method of claim 1, wherein the daily dose of the levetiracetam in the pharmaceutical composition is 220 mg and wherein the pharmaceutical composition further comprises 280 mg-350 mg of hydroxypropyl methylcellulose, 1.2 mg-1.4 mg of colloidal silicon dioxide, 92.8 mg-119.2 mg of silicified microcrystalline cellulose, and 6.0 mg-6.7 mg of magnesium stearate.

22.-24. (canceled)

25. The method of claim 21, wherein the hydroxypropyl methylcellulose is hypromellose 2208.

26. The method of claim 21, wherein the silicified microcrystalline cellulose is silicified microcrystalline cellulose SMCC 90.

27. The method of claim 1, wherein the pharmaceutical composition comprising the levetiracetam, or pharmaceutically acceptable salt thereof, is in a once-a-day extended release form and provides a steady state plasma concentration of levetiracetam in a subject of between 1.9 μg/mL and 4.4 μg/mL within 3 hours after administration and extending for at least 8 hours of a 24-hour period after said administration.

28.-32. (canceled)

33. A method for delaying or reducing the rate of volumetric atrophy of a subregion of the medial temporal lobe in an APOE4 non-carrier subject, the method comprising administering to the subject one or more of levetiracetam, brivaracetam or seletracetam, or a pharmaceutically acceptable salt thereof, wherein the levetiracetam, brivaracetam or seletracetam are administered at a daily dose of 0.7-350 mg, or comprising administering to the subject a pharmaceutical composition comprising the daily dose of the levetiracetam, brivaracetam or seletracetam, or a pharmaceutically acceptable salt thereof, and a pharmaceutically acceptable carrier.

34. The method of claim 33, wherein the subregion of the medial temporal lobe is the entorhinal cortex (ERC) or the transentorhinal cortex (BA35).

35.-39. (canceled)

40. The method of claim 33, wherein the APOE4 non-carrier subjects carry a genetic risk factor for the development of cognitive impairment which is not APOE4.

41. The method of claim 33, wherein the subject presents or displays with cognitive performance below the normal range for their age.

42. The method of claim 33, wherein the subject presents or displays with volumetric atrophy of a subregion of the medial temporal lobe.

43.-47. (canceled)

48. The method of claim 33, wherein the subject presents with or displays pre-mild cognitive impairment, mild cognitive impairment, amnestic mild cognitive impairment (aMCI), mild cognitive impairment due to Alzheimer's disease (AD) or mild cognitive impairment due to prodromal AD.

49.-56. (canceled)

57. The method of claim 33, wherein the pharmaceutical composition is formulated in one or more of an oral form, an extended-release form, a single-unit-dosage form or a once-a-day form.

58.-59. (canceled)

60. The method of claim 33, wherein the daily dose of the levetiracetam in the pharmaceutical composition is 220 mg and wherein the pharmaceutical composition further comprises 280 mg-350 mg of hydroxypropyl methylcellulose, 1.2 mg-1.4 mg of colloidal silicon dioxide, 92.8 mg-119.2 mg of silicified microcrystalline cellulose, and 6.0 mg-6.7 mg of magnesium stearate.

61.-65. (canceled)

66. The method of claim 33, wherein the pharmaceutical composition comprising the levetiracetam, or pharmaceutically acceptable salt thereof, is in a once-a-day extended release form and provides a steady state plasma concentration of levetiracetam in a subject of between 1.9 μg/mL and 4.4 μg/mL within 3 hours after administration and extending for at least 8 hours of a 24-hour period after said administration.

67.-71. (canceled)
